(19) United States
(12) Patent Application Publication
(10) Pub. No.: US 2008/0306576 A1
(43) Pub. Date: Dec. 11, 2008

(54) Optical Cell Control Prosthetics

(75) Inventors: Edward S. Boyden, Cambridge, MA (US); Jacob G. Bernstein, Cambridge, MA (US)

Correspondence Address: NORMA E HENDERSON;HENDERSON PATENT LAW, 13 JEFFERSON DR, LONDONDERRY, NH, 03053, US

(73) Assignee: Massachusetts Institute of Technology, Cambridge, MA (US)

(21) Appl. No.: 12/118,673

(22) Filed: May 9, 2008

Related U.S. Application Data
(60) Provisional application No. 60/917,055, filed on May 9, 2007.

(51) Int. Cl. A61N 5/06

(52) U.S. Cl. 607/91

(57) ABSTRACT

A prosthetic device for optical control of target cells comprises a set of light sources, hardware for guiding light to the target cells, supporting hardware that holds members of the set of light sources with respect to each other and the target cells, control circuitry for controlling the set of light sources, and power circuitry that provides power to the set of light sources and the control circuitry. The device may be wearable or implantable, and may be remotely powered or employ wireless communication. The supporting hardware may comprise implantable hypodermics or cannulas, or a plate or scaffold. The set of light sources may be assembled into an array.

### RELATED APPLICATIONS

This application claims the benefit of U.S. Provisional Application Ser. No. 60/917,055, filed May 9, 2007, the entire disclosure of which is herein incorporated by reference.

### FIELD OF THE TECHNOLOGY

The present invention relates to methods and devices for control of cell function and, in particular, to prosthetic devices for optical control of cells.

### BACKGROUND

Many diseases of the human brain and nervous system are related to dysfunction of specific neuron types, which undergo pathological changes in number, excitability, anatomy, or synaptic connectivity. These changes lead, via altered neural circuit activity, to the perceptual, cognitive, emotional, and motor deficits associated with various neurological and psychiatric illnesses. For example, temporal lobe epilepsy is associated with increased excitability and connectivity of specific excitatory neurons [C. Bernard, A. Anderson, A. Becker et al., Science 305 (5683), 532 (2004); E. R. Sanabria, H. Su, and Y. Yaari, J Physiol 532 (Pt 1), 205 (2001); L. R. Shao and F. E. Dudek, J Neurophysiol 92 (3), 1366 (2004); C. R. Houser, J. E. Miyashiro, B. E. Swartz et al., J Neurosci 10 (1), 267 (1990)] and the loss of specific kinds of inhibitory interneurons [P. S. Buckmaster and F. E. Dudek, J Comp Neurol 385 (3), 385 (1997)] in the hippocampus, whereas schizophrenia is associated with atrophy of a specific kind of inhibitory neuron in the prefrontal cortex [D. A. Lewis, T. Hashimoto, and D. W. Volk, Nat Rev Neurosci 6 (4), 312 (2005)].

The ability to optically activate or inactivate genetically-specified excitable target cells, such as central nervous system neurons, glia, peripheral neurons, skeletal muscle, smooth muscle, cardiac muscle, pancreatic islet cells, thymus cells, immune cells, or other excitable cells, embedded in intact tissue, such as brain, peripheral nervous system, muscle, and skin, would enable radical new treatments for many disorders (e.g., neuropathic pain, Parkinson's disease, epilepsy, diabetes, and other diseases). Molecular-genetic methods for making cells such as neurons sensitive to being activated (e.g., depolarized) or inactivated (e.g., hyperpolarized) by light have been previously developed [X. Han and E. S. Boyden, “Multiple-color optical activation, silencing, and desynchronization of neural activity, with single-spike temporal resolution,” PLoS ONE 2, e299 (2007)], but no method currently exists for delivering light to precise locations in intact tissues.

### SUMMARY

The present invention is a device for delivering light to precise locations in intact tissues, in order to optically activate or inactivate specified excitable target cells. The invention comprises a set of light sources, accessory hardware for guiding light, supporting hardware to hold members of the set of light sources with respect to each other, the target cells, and external structures, and control and power electronics that monitor target cell state, provide regulated power to the light sources, and communicate data, stimulation protocols, and algorithms. The device may be wearable or implantable, and may optionally be remotely powered or employ wireless communication. The set of light sources may be assembled into an array.

In a preferred embodiment, an array of fiber-coupled LED elements are attached to a support. The LED elements are each connected to an optical fiber and a wire. Each wire can run through an optional cannula and are attached to the control circuitry. The target ends of the fibers are aimed to deliver light to specific target cells. In an alternative preferred embodiment, the LED is placed at the tip of a hypodermic or cannula and optionally coated by a biocompatible coating.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 is a diagram depicting a fiber-coupled LED element and an array composed of multiple such elements, according to one aspect of the present invention;

FIG. 2 is a diagram depicting a hypodermic LED source, according to another aspect of the present invention;

FIG. 3 is a diagram depicting an embodiment of a plate for holding the circuitry and LEDs, according to another aspect of the present invention; and

FIGS. 4A-D are diagrams depicting four alternative embodiments of an electronics board for operating the fiber array, according to a further aspect of the present invention.

### DETAILED DESCRIPTION

The present invention is a device for delivering light to precise locations in intact tissues. The invention employs sets of light sources coupled to optical fibers whose ends deliver light to specified groups of target cells within tissue, sets of light sources in hypodermic cannulas that can deliver light locally to specified groups of target cells within tissue, and sets of light sources attached to nerve cuff holding devices that stably bring the light sources into close proximity to a group of target nerve cells. In support of the function of these sets of light sources, the present invention in some aspects includes control and power electronics, which enable battery-powered, wearable, fully implantable, wirelessly-operated, and/or remotely-powered versions of the electronics to drive these light sources, thus enabling the use of these devices as prosthetics. In another aspect, the present invention includes steerable light sources, ways of coupling multiple colors into the same fiber, and other uses of such fibers.

The invention comprises several parts: a set of light sources (such as, but not limited to, LEDs or lasers) with accessory hardware (e.g., fibers) for guiding light, supporting hardware to hold members of the set of light sources with respect to each other, with respect to the target cells such as, but not limited to, brain, glia, peripheral nerve, skeletal muscle, smooth muscle, cardiac muscle, pancreatic islet cells, thymus cells, or other excitable cells, embedded in the tissue (such as, but not limited to, brain, peripheral nervous system, muscle, skin, pancreas, and heart), and perhaps held firm with respect to external structures (such as, but not limited to, skull, skeleton, muscle, or skin), and control and power electronics that monitor target cell state, provide regulated power to the light sources, communicate data, stimulation protocols, and algorithms to and from the outside world, and/or may be remotely powered by external electromagnetic fields or other kinds of wireless energy.

In the present invention, it should be understood by one of ordinary skill in the art that each of the variations on the component parts of the invention are swappable with any of the other variations. Similarly, when a use of the present invention is described with respect to a particular tissue or body part, it will be understood by one of ordinary skill in the art that the invention can be used in a similar manner for other body parts and tissues. For example, if a use is described is for the “brain,” then it may similarly used in any other bodily tissue (e.g., peripheral nerve, pancreas, etc.). As another example, if it is described how to affix something to the skin, it may similarly be used in dealing with muscle and other tissues as well. The terms light source, LED, or laser are also used interchangeably, as they all have similar functionality in the context of the present invention. The light produced by the source may be visible light, infrared, spectrally complex, or any other type of light found to be suitable for the particular application.

A set of light sources is typically employed, although not absolutely required, because tissues are highly scattering, so that in many cases no one light source will be able to illuminate all the target cells in the entire desired target area [M. H. Niemz, Laser-Tissue Interactions: Fundamentals and Applications. (Springer-Verlag Telos, 1996); Bevilacqua, F, Marquet, P, Depeursinge, C, Haller, E B “Determination of reduced scattering and absorption coefficients by a single charge-coupled device array measurement, part II: measurements on biological tissues.” Opt. Eng. 34: 2064-2069 (1995); E Okada, E, Schweiger, M, Arridge, S R, Firbank, M, Delpy, D T, “Experimental validation of Monte Carlo and finite-element methods for the estimation of the optical path length in inhomogenous tissue”, Appl. Opt., 1996. 35: p. 3362-71 (1996)]. Each individual light source must receive electrical power, and deliver light locally to its target cells. In one embodiment, each light source is coupled to an optical fiber that projects deep into the tissue of interest to deliver light to the target cells. The electrical leads of the light source extend to the power/control circuitry, which provides timed pulses of electricity to the light source. The entire set of light sources may comprise many such optical elements, and in a preferred embodiment are arranged in an array on supporting hardware, with all the light sources in a plane, the fibers projecting perpendicularly into a tissue, and the ends terminating in various target regions where the target cells reside. In alternative preferred embodiments, the plane is a flexible substrate, so that fibers project inward from a curved surface into the tissue (e.g., if the target cells are in a tissue that is a naturally curved substrate like the brain), or there are multiple flat planes connected at their edges (e.g., forming part of a polyhedron).

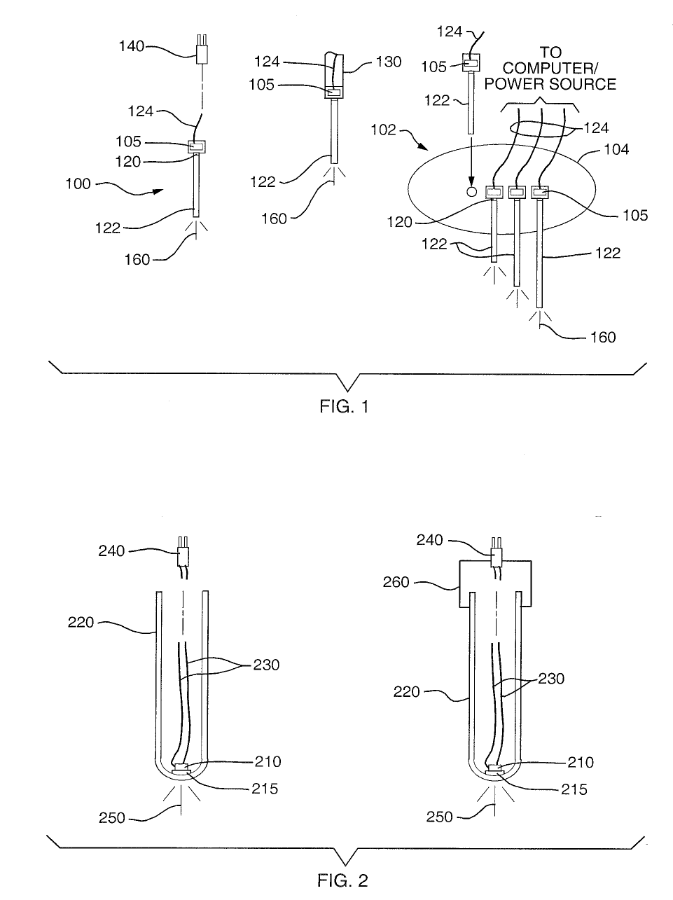

FIG. 1 is a schematic diagram depicting fiber-coupled LED element 100 and array 102 composed of multiple such elements, attached to plate 104, according to one aspect of the present invention. In FIG. 1, LEDs 105 (such as, but not limited to, yellow or blue ones) are shown glued with optical adhesive 120 to optical fibers 122, each with a wire 124 (such as, but not limited to, copper) emerging in the direction opposite to the direction of a fiber 122. The wires can run through optional cannula 130 for protection, strain relief, and biocompatibility, and are optionally attached to electrical socket 140 at the end to provide easy attachment and disconnection. Cannula 130 may be made of any suitable material known in the art including, but not limited to, stainless steel, titanium, or glass. The target ends of the fibers are aimed to deliver light 160 to specific target cells in the target tissue, and can terminate at different depths within it. For example, in the brain, the target cells might be neurons. Array holder plate 102 may be made of any suitable material known in the art including, but not limited to, PCB board, kaptan, or steel. It may also alternatively be a hollow scaffold-type design, rather than being a solid plate.

An alternative preferred implementation of the set of light sources is to place the LED at the tip of a hypodermic or cannula, attached to the walls of the cannula with optical adhesive and optionally coated by a biocompatible coating. FIG. 2 is a diagram depicting a hypodermic LED source, according to this aspect of the present invention. LED 210 is attached by optical adhesive 215 at the aperture of implantable hypodermic 220. In a preferred embodiment, hypodermic 220 is steel, but it may be any suitable material known in the art. Wires 230 attached to LED 210 snake up tube 220, which is optionally attached to connector 240. This has the advantage of delivering light 250 directly to the area of interest, while minimizing fiber-coupling losses. Optional housing 260 may also be used. The need to place a large LED at depth may require more room for the implantation, as opposed to a very small fiber, but as LEDs become smaller and smaller, this difference will become moot. Calculations indicate that tissue heating due to local light generation will be negligible for almost all clinically relevant applications of the LED. The LEDs and hypodermics can optionally be assembled into an array, as shown in FIG. 1.

Another preferred implementation has a bare LED, potted in a biocompatible coating, with wires leading out of the coating. Yet another preferred implementation has the LED on a peripheral nerve cuff (e.g., as used in nerve cuff electrodes), which brings the LED in close apposition to a nerve that is desired to be stimulated. This enables stimulation of peripheral nerves, e.g. for sensory replacement, controlling motor outputs, or silencing pain neurons.

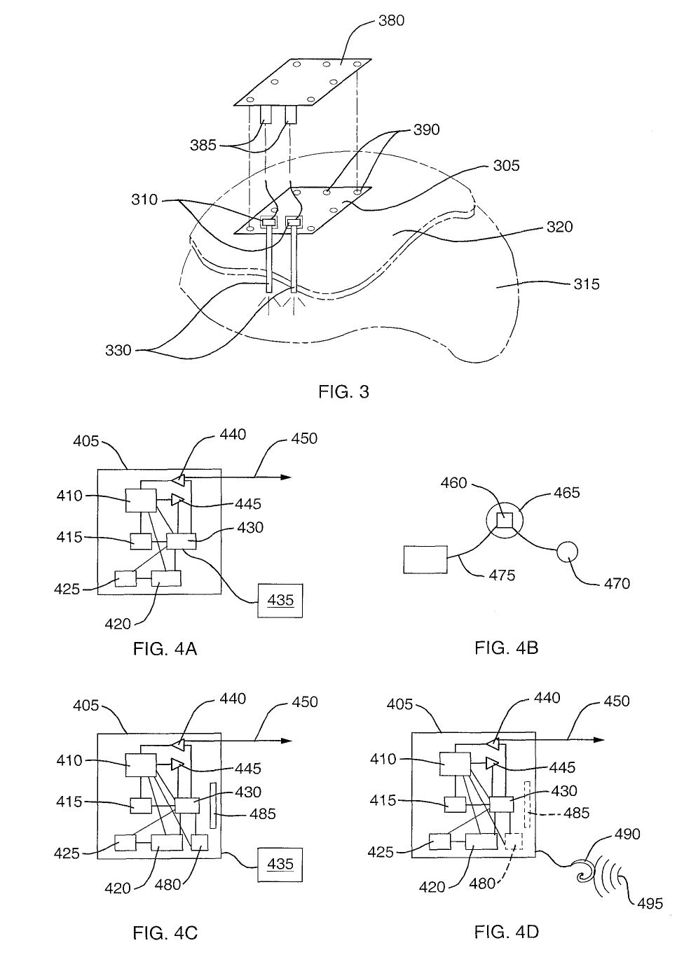

FIG. 3 depicts an embodiment of a plate for holding the circuitry and LEDs, according to one aspect of the present invention. Plane 305 containing all the light sources can correspond to a physical plate made of printed circuit board materials, including, but not limited to, kapton, polyimide, titanium, and stainless steel, that holds LEDs 310 firmly oriented, via adhesive or mechanical fitting into holes, towards the targets on brain 315 and skull 320. Plate 305 may be conformal or may alternatively be of scaffold-type design. LEDs 310 may be connected to optical fibers 330, as in FIG. 1, or within hypodermic tubes, as in FIG. 2. The cannulas, plates, sockets, etc. make up the supporting hardware, which is designed to connect to electronics board 380 (and FIG. 4) via sockets 385 and holes 390 for screws, dental acrylic, or other means known in the art for docking board 305 with the upper layers of the device. The embodiment of FIG. 3 is suitable for use with many different types of implementations, including wearable, implanted, wirelessly-controlled, or remotely-powered implementations. The device of FIG. 3 is capable of being implanted under the skull, within the brain, or in within one or more parts of the body.

In all of the above scenarios, the light source typically has one or more wires emerging from the supporting hardware. These wires lead to the control and power electronics. The wires need not be physical strands; instead, multiple circuit boards can directly dock with one another. The control and power electronics contain all of the elements needed to power the light sources when light is desired, to perform any necessary computations, to communicate with the outside world to obtain light pulse programs or to upload data, to store data locally, to acquire power from remote sources, or to detect local phenomena in the brain circuit (including, but not limited to, spikes or field potentials detected on an electrode) in order to react appropriately and deliver light of the appropriate wavelength, power, timecourse, etc. For example, a particularly appealing way to modulate LED power with a simple circuit is to pulse width modulate (PWM) the LED. A particularly simple wireless method is to simply attach an LED to an inductor, which is then remotely powerable.

Various embodiments of these circuits are battery-powered, wearable, fully implantable, wirelessly-operated, and/or remotely-powered, so different versions of the electronics may be advantageously employed to drive the LEDs. FIGS. 4A-D are diagrams depicting four alternative electronics boards for operating the fiber array, according to this aspect of the present invention. Wearable (FIG. 4A) implementations contain all the computational and power capacity onboard, as do implantable (FIG. 4B) versions. As shown in FIG. 4A, board 405 supports microcontroller 410, preferably with D/A converters (such as, for example, but not limited to, a PIC microcontroller), RAM 415, flash memory 420 to store the pulse program, and USB 425 for uploading programs and downloading data and/or logs. On-board DC power source 430 is supported by battery 435, which is an Li ion battery in a preferred embodiment but could also be any other suitable battery or other power source known in the art including, but not limited to, an ultra capacitor or even a wall connection. Board 405 also supports amplifiers 440, 445 or other circuits to drive the LEDs and electrodes or other neural sensors 450, which provide information that can permit microcontroller 410 to trigger light pulses in a dynamic way. As shown in FIG. 4B, LED 460 is embedded in biocompatible coating 465, powered by battery 470, and is connected 475 to a board that is similar to, or the same as board 405 from FIG. 4A.

Wirelessly-operated devices, such as the one shown in FIG. 4C, are implemented like the wearable and implantable devices of FIGS. 4A and 4B, but they also comprise transceiver 480 and antenna 485 in order to receive and transmit information via RF. While RF transceiving is described, it will be clear to one of ordinary skill in the art that any kind of wireless communication may be advantageously employed in the present invention, including, but not limited to, ultrasound and optical, each of which have associated specialized hardware requirements. Remotely-powered devices, such as the one shown in FIG. 4D, require antenna 490, specialized for the capture of magnetic or RF energy 495, such as, but not limited to an inductor, power RF coil, or RFID chip. They can also be wireless, like the embodiment of FIG. 4C by incorporating transceiver 480 and antenna 485. Depending on the disorder being treated, the duration of the treatment, and the risks associated with various kinds of implant, various subsets or combinations of these specifications may be found to be desirable for a particular individual patient.

In an example implementation, specific to the brain and skull, materials used include unjacketed optical fiber—100 μm, 200 μm, or 500 μm UV-VIS transmitting (FIG. 1), ultra-thin wall stainless steel hypodermic tubing, ultrabright blue LEDs (e.g., EZ1000 for coupling to fibers (FIG. 1) or EZ290 used for coupling to fibers or being implanted directly in brain in hypodermic (FIG. 2)), ultrabright yellow LEDs (Luxeon III, Luxeon Rebel, used for coupling to fibers (FIG. 1), Lumileds P4—implanted directly in brain in hypodermic (FIG. 2)), and optical adhesive. Tools required include UV curer, Dremel, water jet cutter, laser cutter, excimer laser, and 3-D printer. The fiber array is made up of two components—the supporting hardware (FIG. 3), and a collection of modular light guides (FIGS. 1 and 2). For small structures, 100 μm and 200 μm optical fiber light guides may be used (FIG. 1), whereas for larger structures, hypodermic light guides may be used (FIG. 2).

For assembly of this example implementation, the lowest layer of the supporting hardware is cut on an excimer laser, with holes for screws to attach the supporting hardware to the skull. The second layer of the supporting hardware screws or pops onto the first, and is a printed circuit board, containing a wireless transceiver, an embedded antenna, a programmable IC, and circuitry to drive current through the LEDs in the light guides (FIG. 3). It interfaces with each light guide through a custom plug. Each light guide has a clearance hole in both layers, as well as docking holes for the housing in the first layer. The light guides are encased in a custom 3D printed housing. At the top of each light guide is a socket to interface with the electronics on the supporting hardware. At the bottom of each light guide is an opening for the optical fiber or LED to emerge. Steel cannulas are cut circumferentially with a Dremel to avoid collapsing or crimping the tubing. For the optical fiber light guides (FIG. 1), the LED sits inside of the housing and is coupled directly to the fiber with optical adhesive, generating light that is sent down the optical fiber, which is implanted directly in the brain. For the hypodermic light guide (FIG. 2), a thin walled stainless steel tube is wired with a 300 μm wide LED at the base, which shines light directly into the brain. Any exposed wire is insulated with biocompatible coating. This particular implementation can shine light about 0.5-1 mm away from atypical fiber (diameter 0.2-0.5 mm).

Currently these fiber arrays are being implemented using individual lasers or LEDs, but arrays of vertical cavity surface emitting lasers (VCSELs) or other optical sources work just as well. It is further envisioned that if, in the future, xenon bulbs, halogen lamps, incandescent bulbs, or other light sources become miniaturized enough to fit, they may also be advantageously used in the prosthetics of the present invention (likely with filters on the bulbs), although current embodiments of these devices are not as viable as LEDs and lasers due to their wasted energy, expense, danger, and limited life.

An optional enhancement is the use of a dichroic (or beamsplitter, or other equivalent optical part) attached to a fiber in a way so that it couples two different light sources (e.g., a blue LED and a yellow LED, or a blue laser and a yellow laser) into the fiber, so that the target cells at the end of the fiber can be activated and deactivated by two different colors of light (see, e.g., X. Han and E. S. Boyden, “Multiple-color optical activation, silencing, and desynchronization of neural activity, with single-spike temporal resolution”, PLoS ONE 2, e299 (2007)). Also suitable is a series of cascaded dichroics, capable of coupling more than two colors of light into the same fiber. Another optional enhancement is a steerable element (such as, but not limited to, a galvanometer, an acousto-optic deflector, a MEMS mirror, or other steering device), on one or both ends of the fiber, in order to direct light in a controlled way, enabling locally selective targeting of the light to specific areas of the tissue, preferably with as few moving parts as possible.

While the present invention has been described in the context of the use of light to excite and inhibit electrically excitable cells, it will be understood by one of skill in the art that the present invention may also be advantageously employed to deliver light to other realms, such as to drive the production of cAMP in deep tissue [Schröder-Lang S, Schwärzel M, Seifert R, Strünker T, Kateriya S, Looser J, Watanabe M, Kaupp U B, Hegemann P, Nagel G. “Fast manipulation of cellular cAMP level by light in vivo”, Nature methods (2006)], to simulate the action of a G-protein coupled receptor acting drug [J. M. Kim, J. Hwa, P. Garriga et al., “Light-driven activation of beta 2-adrenergic receptor signaling by a chimeric rhodopsin containing the beta 2-adrenergic receptor cytoplasmic loops”, Biochemistry 44 (7), 2284 (2005)], or to change the pH of a cell [G. Nagel, D. Ollig, M. Fuhrmann et al., “Channelrhodopsin-1: a light-gated proton channel in green algae”, Science 296 (5577), 2395 (2002)]. There are many therapeutic reasons to desire these abilities.

While a preferred embodiment is disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. A prosthetic device for optical control of target cells, comprising:
a set of light sources;
lightguide hardware, connected to the light sources, for guiding light to the target cells;
supporting hardware that holds members of the set of light sources with respect to each other and the target cells;
control circuitry for controlling the set of light sources; and
power circuitry that provides power to the set of light sources and the control circuitry.

2. The device of claim 1, further comprising circuitry for wireless communication.

3. The device of claim 1, wherein the power circuitry comprises a battery.

4. The device of claim 1, wherein the power circuitry is remotely powered.

5. The device of claim 1, wherein the supporting hardware comprises implantable hypodermics or cannulas.

6. The device of claim 1, wherein the supporting hardware comprises a plate or scaffold.

7. The device of claim 6, wherein the set of light sources is assembled into an array.

8. The device of claim 1, wherein the light sources are embedded in a biocompatible coating.

9. The device of claim 1, further comprising sensors for monitoring the target cells.

10. An array of prosthetic devices for optical control of target cells, each prosthetic device comprising:
a light source;
lightguide hardware, connected to the light source, for guiding light to at least one target cell;
supporting hardware that holds the prosthetic device with respect to other prosthetic device and the target cells;
control circuitry for controlling the light source; and
power circuitry that provides power to the light sources and the control circuitry.

11. The array of claim 10, further comprising circuitry for wireless communication.

12. The array of claim 10, wherein the power circuitry is battery powered.

13. The array of claim 10, wherein the power circuitry is remotely powered.

14. The array of claim 10, wherein the supporting hardware comprises an implantable hypodermic or cannula.

15. The array of claim 10, wherein the supporting hardware comprises a shared plate or scaffold.

16. The array of claim 10, wherein the array is embedded in a biocompatible coating.

17. The array of claim 10, further comprising sensors for monitoring the target cells.
